(19) United States
(12) Patent Application Publication
(10) Pub. No.: US 2022/0211532 A1
(43) Pub. Date: Jul. 7, 2022

(54) RLJ 2 Point Brace Non-Weight Bearing Cast

(71) Applicant: Ricky L. Jones, Choctaw, OK (US)

(72) Inventor: Ricky L. Jones, Choctaw, OK (US)

(21) Appl. No.: 17/142,236

(22) Filed: Jan. 5, 2021

(51) Int. Cl. A61F 5/01; A61F 5/30

(52) U.S. Cl. CPC A61F 5/0111; A61F 2005/0188; A61F 5/30

(57) ABSTRACT

Methods for installing brackets that protect the heel of a person wearing a non-loadbearing cast. In one embodiment two brackets are attached at two points on opposite sides of the foot, with one bracket below the heel and the other behind the heel with roughly 0.75 inch clearance. This arrangement serves to redirect a load that would have been applied to the heel. The loads are now directed to either side of the ankle at the two attachment points.

This application claims the benefit of provisional application of the same name filed Jan. 3, 2020

### BACKGROUND OF THE INVENTION

The problem with loads on non weightbearing casts is that the flesh of the heel easily develops ulcers when contact over 5 pounds is applied. There are no notable solutions to this problem in the prior art.

This inventor arrived at this design as an orthopedist, in a Technician capacity, over the course of 30 plus years of experience. It is a necessary improvement to the art of cast making.

This disclosure provides the method for installing the apparatus, and similar competitive conceptual apparatuses.

### BRIEF SUMMARY OF THE INVENTION

This disclosure provides a method and alternate method embodiments for installing an apparatus, which can be used to install similar competitive conceptual apparatuses.

About the Apparatus and Similar Conceptual Apparatuses

The solution herein disclosed redirects the two common axial forces from the heel area to a point above and forward. The primary embodiment includes two brackets that protect the bottom and back of the heel from contacts. The brackets are cast as part of the leg cast with plaster or other methods.

The first alternative embodiment comes from the objective of the device—to prevent contact at the heel. The idea is to add an audible “click” at the bracket to notify the wearer that a load of more than 5 pounds has been experienced by the cast. The click should reset—spring loaded perhaps—so that it is repetitive, notifying the wearer of his/her many 5 #overloads. The intent is to be disruptive, to create an incentive to stop overloading the cast.

The second alternative embodiment speaks to children—who are probably notorious for overloading the cast at the heel. The suggestion is to reshape the attached brackets to a more entertaining but functional design. Replacing the rear and lower guards with a pair of ears and a tail, for example.

Another embodiment continues to use electronics that would allow both notice that an overload has occurred and there is an aesthetic advantage for younger wearers.

a. in an embodiment, Electronics includes switches mounted on the brackets to activate on 5 #overload, with red LED indicators that flash for 5 seconds when an overload condition is momentarily present.

b. Electronics can include a sonolert—an audible alarm to indicate an overload condition. The alarm can be a click, for subtlety. The alarm can be a garish siren.

In an embodiment, Other electronics include a molded pressure pad with multiple pressure sensors that respond to 5 #loading. Schmersal sms4-500-500 is an exemplary pressure mat with about 244 sensors. This would be overkill, but offers the concept of a low profile mat with multiple input sensors. In this application, only 4 to 6 sensors are needed in a minimal arrangement.

In an embodiment, in place of the brackets, a single molded cap could be used. The cap would secure to the cast similar to the brackets—at two opposite sides of the heel. The cap, however would be closer to the cast than ¾″. The cap can have electronic switches that mount and operate similar to those previously discussed. A concept is below, the already broken nose does not stop a player from basketball because the mask redirects any forces away from the nose and to other parts of the face.

In an embodiment, using the primary embodiment concept, it is possible to redirect forces to any best part of the cast using dual mounts and eccentric loading. That is to say, the original design directs all forces to the two points of attachment. If, in fact, the best place to redirect a load is to the back of the ankle—along the achilles. Were that true, the rear bracket could attach at two points on either side of the ankle—4 points total. On each side, the first attachment point is lower than the other—one toward the sole of the foot and the other higher on the leg—perhaps ½″ apart. The attachment points are also slightly (e.g. ¼″) offset forward and rearward. With this 4 point mounting, any load directed toward the back of the heel would be received by the two attachment points unevenly. This would cause a torque that would apply the force at the back of the leg or on the sole of the foot. With an alternative positioning of the 4 mounting points a torque could be applied wherever is best.

In an embodiment, force pads (a pair of pads electronically connected by a normally open switch) are used as contact points molded into the cast by applying an additional pad at the places where the load is more optimally applied. The concept of a broad, soft pad at the heel was rejected because it could force the contact and a resulting ulcer. However, the top of the foot, the bottom sole of the foot and the back of the ankle are apparently good places to direct/redirect forces. Here, during the cast molding process a force pad is placed in contact with the foot/leg at the best points for directing contact forces—and then the remaining wrapping and other casting processes are completed.

The electronic concepts apply to all embodiments presented.

I claim:

1. A method for installing a spacer to orthopedic casts comprised of the following steps
a doctor or provider places the order for orthopedic technicians to apply a cast to a patient for use with nonweightbearing casts including:
Fiberglass long leg cast,
short leg cast,
Fiberglass long leg splints,
short leg splints.
Plaster long leg cast,
short leg cast,
Plaster long leg splints,
short leg splints.
Hip Spica Cast
long leg Fiberglass and Plaster,
And similar types of casts;
to aid in healing the following common fractures:
Tibia, Fibula, Ankle, Navicular, Cuboid, Cuneiform Bones, Stress Fractures, etc;
the Orthopedic Technician applies the appropriate nonweightbearing cast;
the Orthopedic Technician opens the packaging for the RJ 2 Point Brace which includes at a minimum, the brace assembly (or two braces) and their attachment hardware;
the user is made aware that the objective is to protect the heel from contact when a force is applied to the cast at the heel area by redistributing the force to the distal and proximal ends of the foot and ankle structures;
the braces are attached to the cast, during or after the cast is completely formed;
the technician places the braces:
a first brace is to be located at the distal end of the foot and cast, with attachments at the inner side of the navicular and outer side of the cuboid bones.
a second brace is to be located to the posterior end of the foot and cast, with attachments at the inner side of the medial malleolus bone and outer side of the lateral malleolus bone;
once the braces are placed, hold the brace in place and secure using 2 inch or 3 inch width fiberglass cast material,
smooth and mold the cast material to secure the braces firmly and smooth edges for uniformity;
once both braces are secured and molded into place, specific care instructions are necessary, including:
a. Even with the braces, this is still a non weight bearing cast. The braces are to be rated at specifically tested and approved weights, for example “no more than a 5 pound load”.
b. Generally, do not get the cast wet. In an embodiment, a waterproof cast liner is used and the water instruction for such a cast will also apply to the bracket
c. Do not insert (“stick”) anything sharp inside the cast
d. Do not remove any of the padding
e. Do not stand on the cast with entire bodyweight
f. And any other reasonable and necessary medical matters of concern;
once patient is clearly informed of the necessary care, inform them of the benefits of the braced cast over the nonbraced cast including: sleeping, resting, living a more normal life by having increased ability to place foot in usual circumstances (as opposed to having to remain in a controlled environment to eliminate possible contact with the cast and other items) such as: attend school during recovery, attend more outside events, go out to restaurants, ride in an automobile, go to movie, shop and any other activities beyond historical restrictions; and give traditional instructions including:
When resting, sitting or sleeping a towel or pillow may be placed below the cast;
When showering or bathing the cast and braces must be covered with plastic and cast covers as provided by your orthopedic tech or other medical professional;
follow their instruction carefully.

2. The method of claim 1 wherein the technician further installs and starts sonolerts, lights, pressure sensors and batteries to enable sensing and feedback during use of the braces.

3. The method of claim 1 wherein the technician further installs decorative elements to improve the appearance and thereby the consistent use and functionality of the braces.

4. The method of claim 1 wherein the technician further installs a rigid shroud that encompasses the heel and protects the cast from direct impact in the heel area, such installation including securing the shroud with fasteners to maintain a gap between all portions of the cast and all portions of the shroud, except for necessary contact at the sides for securing one to the other.
